(19) United States
(12) Patent Application Publication
(10) Pub. No.: US 2026/0027534 A1
(43) Pub. Date: Jan. 29, 2026

(54) REACTOR SYSTEM FOR ACETYLENE ABSORPTION AND SELECTIVE HYDROGENATION

(71) Applicant: Chevron U.S.A. Inc., San Ramon, CA (US)

(72) Inventors: Huping Luo, Moraga, CA (US); Xiaoying Ouyang, Albany, CA (US); Christopher Declan Lane, El Cerrito, CA (US); Howard Steven Lacheen, Richmond, CA (US)

(21) Appl. No.: 19/214,447

(22) Filed: May 21, 2025

Related U.S. Application Data
(60) Provisional application No. 63/674,413, filed on Jul. 23, 2024.

(51) Int. Cl. B01J 8/02; B01D 53/14; B01D 53/18; B01J 38/04; C07C 5/09

(52) U.S. Cl. CPC B01J 8/0285; B01D 53/1487; B01D 53/18; B01J 8/0242; B01J 8/0278; B01J 38/04; C07C 5/09; B01D 2257/7022; B01J 2208/00176; B01J 2208/00539; B01J 2208/00805; B01J 2208/021

(57) ABSTRACT

A system including an absorption column configured to receive an acetylene-rich gas stream flowing upwards and a cooled acetylene-lean solvent stream flowing downwards to generate an acetylene-lean gas effluent and an acetylene-rich solvent effluent, one or more heat exchangers for receiving the acetylene-rich solvent effluent, the one or more heat exchangers being configured to transfer heat to the acetylene-rich solvent effluent to generate a heated acetylene-rich solvent stream, and one or more hydrogenation reactors each having one or more catalyst beds, wherein at least a first one of the one or more hydrogenation reactors is configured to convert at least a portion of acetylene in the heated acetylene-rich solvent stream to ethylene in the presence of a first hydrogenation catalyst and hydrogen under first hydrogenation reaction conditions to generate a first hydrogenation effluent including ethylene and a first acetylene-lean solvent effluent.

### PRIORITY CLAIM

The present application claims priority to U.S. Provisional Patent Application Ser. No. 63/674,413, entitled “Reactor System for Acetylene Absorption and Selective Hydrogenation,” filed Jul. 23, 2024, the content of which is incorporated by reference herein in its entirety.

### BACKGROUND

The ongoing search for alternatives to crude is increasingly driven by a number of factors. These include diminishing petroleum reserves, higher anticipated energy demands, and heightened concerns over greenhouse gas emissions from sources of non-renewable carbon. In view of its abundance in natural gas reserves, as well as in gas streams obtained from biological sources (biogas), natural gas has become the focus of a number of possible routes for providing liquid hydrocarbons. Natural gas occurs underground and is present as a gas when it comes out of the ground. Natural gas primarily consists of methane (CH₄), and additionally some other hydrocarbons such as ethane (C₂H₆) and propane (C₃H₈). Accordingly, converting light hydrocarbons such as methane to high value products such as hydrogen, olefins and aromatics has become an attractive option.

### SUMMARY

In accordance with an illustrative embodiment, a system comprises:
an absorption column configured to receive an acetylene-rich gas stream flowing upwards and a cooled acetylene-lean solvent stream flowing downwards to generate an acetylene-lean gas effluent and an acetylene-rich solvent effluent,
one or more heat exchangers in fluid communication with the absorption column for receiving the acetylene-rich solvent effluent, the one or more heat exchangers being configured to transfer heat to the acetylene-rich solvent effluent to generate a heated acetylene-rich solvent stream, and
one or more hydrogenation reactors, in fluid communication with the one or more heat exchangers, each comprising one or more catalyst beds, wherein at least a first one of the one or more hydrogenation reactors is configured to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

In accordance with another illustrative embodiment, a continuous process comprises:
passing an acetylene-rich gas stream to an absorption column flowing upwards and a cooled acetylene-lean solvent stream flowing downwards to generate an acetylene-lean gas effluent and an acetylene-rich solvent effluent,
passing the acetylene-rich solvent effluent through one or more heat exchangers to transfer heat to the acetylene-rich solvent effluent and generate a heated acetylene-rich solvent stream, and
passing the heated acetylene-rich solvent stream to at least a first one of one or more hydrogenation reactors each comprising one or more catalyst beds to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

In accordance with another illustrative embodiment, a continuous process comprises:
receiving, in a heat exchanger, an acetylene-rich solvent effluent from an absorption column, and a first acetylene-lean solvent effluent from a hydrogenation reactor to transfer heat from the first acetylene-lean solvent effluent to the acetylene-rich solvent effluent and generate a heated acetylene-rich solvent stream and a cooled acetylene-lean solvent stream, and
passing the heated acetylene-rich solvent stream to the hydrogenation reactor comprising one or more catalyst beds to convert at least a portion of acetylene in the heated acetylene-rich solvent stream to ethylene in the presence of a hydrogenation catalyst and hydrogen under hydrogenation reaction conditions to generate a hydrogenation effluent comprising ethylene and a second acetylene-lean solvent effluent,
wherein the first acetylene-lean solvent effluent and the second acetylene-lean solvent effluent from the hydrogenation reactor and the acetylene-rich solvent effluent from the absorption column operate in a continuous solvent recycle loop.

### BRIEF DESCRIPTION OF THE DRAWINGS

In combination with the accompanying drawings and with reference to the following detailed description, the features, advantages, and other aspects of the implementations of the present disclosure will become more apparent, and several implementations of the present disclosure are illustrated herein by way of example but not limitation. The principles illustrated in the example embodiments of the drawings can be applied to alternate processes and apparatus. Additionally, the elements and features shown in the drawings are not necessarily to scale, emphasis instead being placed upon clearly illustrating the principles of the example embodiments. Certain dimensions or positions may be exaggerated to help visually convey such principles. In the drawings, the same reference numerals used in different embodiments designate like or corresponding, but not necessarily identical, elements. In the accompanying drawings:

FIG. 1A illustrates a schematic diagram of a system and process for the selective hydrogenation of acetylene in an acetylene-rich solvent stream to ethylene with a co-current flow of the acetylene-rich solvent stream and a hydrogen stream in an acetylene hydrogenation reactor operating with a continuous solvent recycle loop, according to an illustrative embodiment.

FIG. 1B illustrates a schematic diagram of a system and process for the selective hydrogenation of acetylene in an acetylene-rich solvent stream to ethylene with a countercurrent flow of the acetylene-rich solvent stream and a hydrogen stream in an acetylene hydrogenation reactor operating with a continuous solvent recycle loop, according to an illustrative embodiment.

FIG. 1C illustrates a schematic diagram of a system and process for the selective hydrogenation of acetylene in an acetylene-rich solvent stream to ethylene in a plurality of acetylene selective hydrogenation reactors operated in parallel with a continuous solvent recycle loop, according to an alternative illustrative embodiment.

### DETAILED DESCRIPTION

Various illustrative embodiments described herein are directed to systems and processes for converting an acetylene-rich gas stream into a hydrogenation effluent comprising ethylene.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and processes are described in terms of “comprising” various components or steps, the systems and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “continuous” as used herein shall be understood to mean a system that operates without interruption or cessation for a period of time, such as where reactant(s) and catalyst(s) are continually fed into a reaction zone and products are continually or regularly withdrawn without stopping the reaction in the reaction zone.

A “fresh catalyst” as used herein denotes a catalyst which has not previously been used in a catalytic process.

A “spent catalyst” as used herein denotes a catalyst that has less activity at the same reaction conditions (e.g., temperature, pressure, inlet flows) than the catalyst had when it was originally exposed to the process. This can be due to a number of reasons, several non-limiting examples of causes of catalyst deactivation are coking or carbonaceous material sorption or accumulation, steam or hydrothermal deactivation, metals (and ash) sorption or accumulation, attrition, morphological changes including changes in pore sizes, cation or anion substitution, and/or chemical or compositional changes.

A “regenerated catalyst” as used herein denotes a catalyst that had become spent, as defined above, and was then subjected to a process that increased its activity to a level greater than it had as a spent catalyst. This may involve, for example, reversing transformations or removing contaminants outlined above as possible causes of reduced activity. The regenerated catalyst typically has an activity that is equal to or less than the fresh catalyst activity.

The term “zone” can refer to an area including one or more equipment items and/or one or more sub-zones. Equipment items can include one or more reactors or reactor vessels, absorption units, separation vessels, distillation towers, heaters, heat exchangers, pipes, pumps, compressors, and controllers. Additionally, an equipment item, such as a reactor, dryer, or vessel, can further include one or more zones or sub-zones.

The term “effluent” refers to a stream that is passed out of a reactor, a reaction zone, or an absorption unit following a particular reaction or separation. Generally, an effluent has a different composition than the stream that entered the reactor, reaction zone, or absorption unit. It should be understood that when an effluent is passed to another component or system, only a portion of that effluent may be passed. For example, a slipstream may carry some of the effluent away, meaning that only a portion of the effluent may enter the downstream component or system.

The term “primarily” shall be understood to mean an amount greater than 50%, e.g., 50.01 to 100%, or any range between, e.g., 51% to 95%, 75% to 90%, at least 60%, at least 70%, at least 80%, etc.

For any FIGURE shown and described herein, one or more of the components may be omitted, added, repeated, and/or substituted. Additionally, it should be understood that in certain cases components of the example systems can be combined or can be separated into subcomponents. Accordingly, embodiments shown in a particular FIGURE should not be considered limited to the specific arrangements of components shown in such FIGURE. Further, if a component of a FIGURE is described but not expressly shown or labeled in that FIGURE, the label used for a corresponding component in another FIGURE can be inferred to that component. Conversely, if a component in a FIGURE is labeled but not described, the description for such component can be substantially the same as the description for the corresponding component in another FIGURE.

Direct conversion of light hydrocarbons such as methane (CH₄), ethane and propane under non-oxidative conditions can produce higher molecular weight hydrocarbons, such as olefins, alkynes and aromatics (e.g., benzene), as value-added chemicals and at the same time produce hydrogen that can be used to make, for example, clean and zero carbon fuel. Hydrogen is one of the more important options for future clean energy. However, the desired product selectivity obtained from the direct conversion processes will depend on the particular type of catalyst as well as reaction condition. In general, this reaction is highly endothermic with an enthalpy of about 90 kJ/mol of CH₄ or 60 kJ/mol of H₂, and the exact value of the reaction heat will depend on the desired product distribution. It is also an equilibrium limited reaction, and high temperatures are usually required to achieve a CH₄ conversion that would be practical for commercial applications. For example, to be commercially practical, maintaining a reactor at a temperature range of 600° C. to 1200° C. is required to achieve an acceptable methane conversion.

The required heat creates other practical challenges. For example, under such temperature conditions, the production of coke or solid carbon in the reactor becomes common, which can significantly reduce the yield of high value products, as well as cause significant operational issues such as plugging of the reactor and catalyst deactivation. Such high temperatures also can require expensive materials for the reactor and can make the design of the reactor challenging.

As a further example, under such high temperature conditions, a substantial amount of acetylene will also be produced during the conversion process along with minor amounts of higher triple bond species and dienes such as C₃H₄. Acetylene and ethylene are hydrocarbons with the formula C₂H₂ and C₂H₄, respectively. They are widely used in the chemical industry, and their worldwide production exceeds that of any other organic compound. In the United States and Europe alone, approximately 90% of ethylene is used to produce ethylene oxide, ethylene dichloride, ethyl benzene and polyethylene. On the other hand, while acetylene could be a high value final product, it is highly unstable. Therefore, separating and purifying acetylene to meet acetylene product specifications could significantly complicate the overall process. As a result, producing ethylene is more desirable than producing acetylene.

In view of these challenges, there is a need for solutions to handle acetylene produced during the conversion process to reduce the associated safety risk as well as simplify the overall process. In addition, it would be advantageous for the reactor design for this process to have the capability to (1) provide the reaction heat needed to maintain an optimized temperature profile to achieve high conversion and (2) regenerate and recycle the catalyst being used. It would further be advantageous if such solutions are more energy efficient than existing approaches to produce hydrogen and value-added chemicals.

Illustrative embodiments address the above and other issues with existing reactor systems and processes for converting an acetylene-rich gas stream into a hydrogenation effluent comprising ethylene. The systems and processes provide many advantages, examples of which are mentioned herein. For example, the non-limiting illustrative embodiments described herein overcome the drawbacks discussed above by providing reactor systems and processes for converting a high concentration of acetylene-rich gas stream into a hydrogenation effluent comprising ethylene at high conversion and high selectivity. Specifically, the non-limiting illustrative embodiments described herein are directed to systems and processes to selectively absorb acetylene from a mixed gas feedstock containing at least a high concentration of acetylene, ethylene, hydrogen, and other light hydrocarbon gases, followed by converting the absorbed acetylene into ethylene, to achieve high acetylene conversion and high ethylene selectivity.

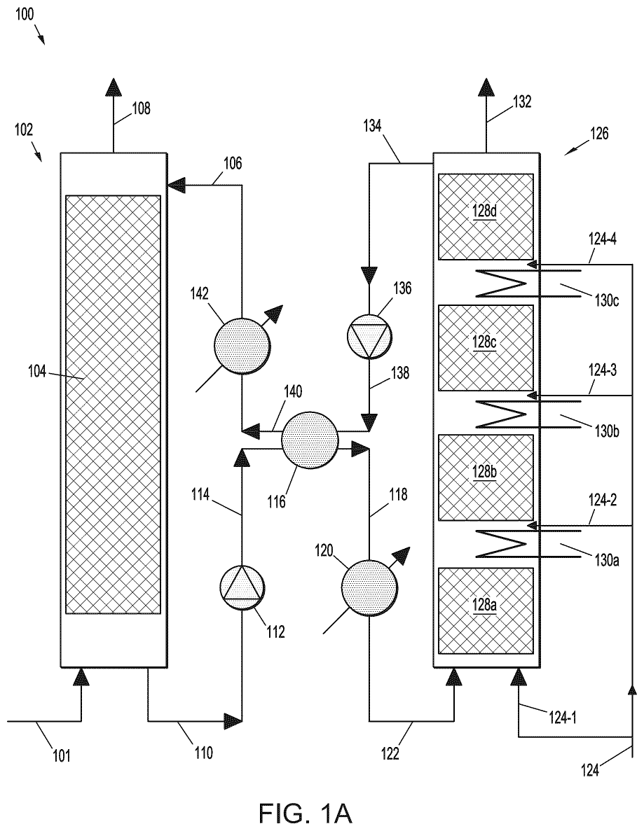

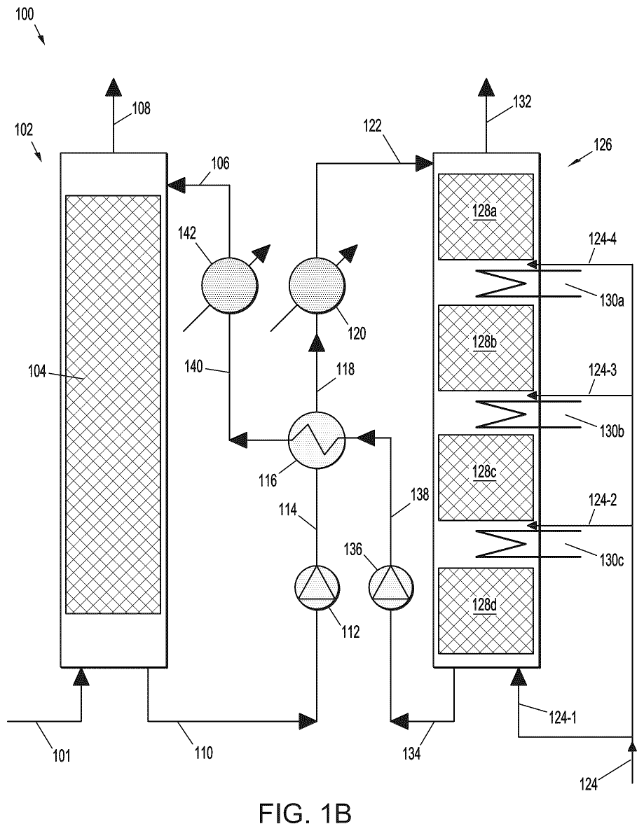

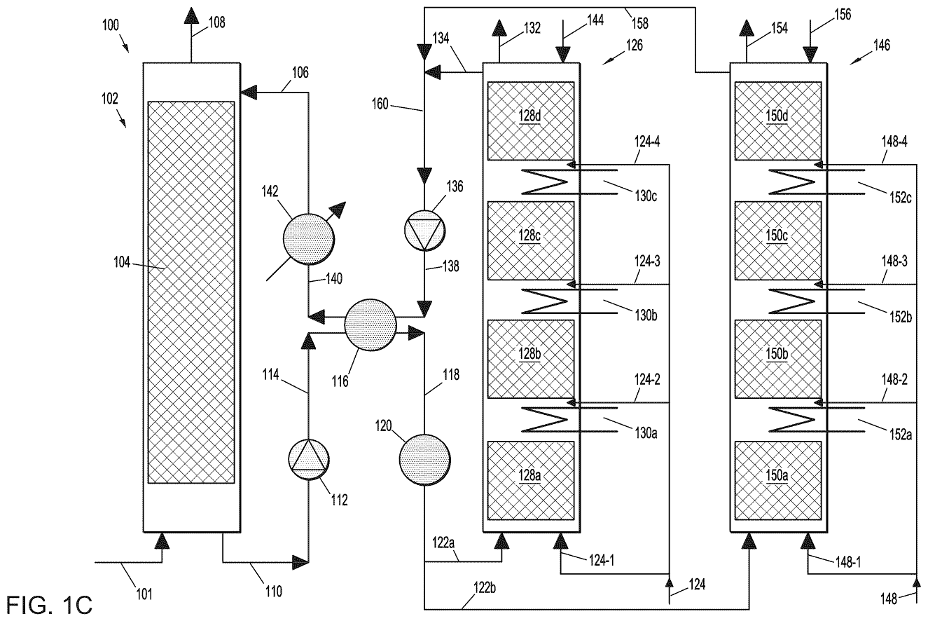

The non-limiting illustrative embodiments of the present disclosure will be specifically described below with reference to the accompanying drawings. For the purpose of clarity, some steps leading up to the production of the hydrogenation effluent comprising ethylene as illustrated in FIGS. 1A-1C may be omitted. In other words, one or more well-known processing steps which are not illustrated but are well-known to those of ordinary skill in the art have not been included in the figures. This is not intended to be interpreted as a limitation of any particular embodiment, or illustration, or scope of the claims.

Referring now to the drawings in more detail, FIGS. 1A-1C illustrate details of systems and processes for improved production of a hydrogenation effluent comprising ethylene from hydrogenation of acetylene utilizing systems including at least an acetylene absorption unit, an acetylene hydrogenation reactor(s) (also referred to as hydrogenation reactor(s), a plurality of heat exchangers and a plurality of pumps. It is to be understood that the system including at least the acetylene absorption unit, the acetylene hydrogenation reactor(s), the plurality of heat exchangers and the plurality of pumps is not limited to the configuration of the embodiments shown in FIGS. 1A-1C, and other configurations are contemplated herein.

Referring now to FIG. 1A, a system 100 includes an acetylene absorption unit 102 for receiving an acetylene-rich gas stream 101 in a bottom portion of acetylene absorption unit 102 and a cooled pressurized acetylene-lean solvent effluent 106 in a top portion of acetylene absorption unit 102 as part of a continuous solvent recycle loop. However, these entry points are merely illustrative and any point of entry of acetylene-rich gas stream 101 and cooled pressurized acetylene-lean solvent effluent 106 into acetylene absorption unit 102 is contemplated. In addition, although it is shown that acetylene-rich gas stream 101 runs countercurrent to cooled pressurized acetylene-lean solvent effluent 106 in acetylene absorption unit 102, it is contemplated that other modes of flow can be carried out such as acetylene-rich gas stream 101 running co-current to cooled pressurized acetylene-lean solvent effluent 106 or combinations of co-current and countercurrent for acetylene-rich gas stream 101 and cooled pressurized acetylene-lean solvent effluent 106.

In some embodiments, acetylene-rich gas stream 101 is obtained from the pyrolysis of a light hydrocarbon feed stream comprising methane such as, for example, natural gas. However, this is merely illustrative and any industrial process for producing an acetylene-rich gas stream is contemplated herein. In some embodiments, acetylene-rich gas stream 101 can contain at least a high concentration of acetylene as well as ethylene, methane, hydrogen and other light hydrocarbons. In some embodiments, acetylene-rich gas stream 101 can contain from about 0.1 wt. % to about 5 wt. % of acetylene.

In some embodiments, cooled pressurized acetylene-lean solvent effluent 106 is a liquid stream containing at least a suitable solvent that has high selectivity to acetylene and low solubility for ethylene. Suitable solvents include liquid solvents such as, for example, aprotic polar solvents. In some embodiments, suitable aprotic polar solvents include, for example, N-alkylated lactam solvents, amide solvents, ketone solvents and the like and combinations thereof. Suitable N-alkylated lactam solvents include, for example, N-methyl-2-pyrrolidone, 1-ethyl-2-pyrrolidone, 1-propyl-2-pyrrolidone (branched or straight chained), or 1-butyl-2-pyrrolidone (branched or straight chained), and the like and mixtures thereof. Suitable amide solvents include, for example, dimethylacetamide dimethylformamide, and the like and mixtures thereof. Suitable ketone solvents include, for example, acetone, and the like, or combinations thereof.

In some embodiments, acetylene absorption unit 102 includes an acetylene absorption column or tower containing a suitable structured or unstructured absorption packing material. Acetylene absorption unit 102 further includes an acetylene absorption zone 104 where at least a portion of acetylene in acetylene-rich gas stream 101 is absorbed by the liquid solvent in cooled pressurized acetylene-lean solvent effluent 106 to produce an acetylene-lean gas effluent 108 and an acetylene-rich solvent effluent 110. Acetylene-lean gas effluent 108 is a vapor stream that is lean in acetylene and comprises hydrogen gas. Acetylene-lean gas effluent 108 exits through a top portion of acetylene absorption unit 102 for further processing.

Acetylene-rich solvent effluent 110 is a liquid stream containing acetylene and solvent. In some embodiments, a concentration of acetylene in acetylene-rich solvent effluent 110 can range from about 0.01 wt. % to about 5 wt. %, or from about 0.1 wt. % to about 2 wt. %.

Acetylene-rich solvent effluent 110 exits through a bottom portion of acetylene absorption unit 102 and is sent to a pump 112. Pump 112 can be any suitable pump for increasing the pressure of acetylene-rich solvent effluent 110 for sending a pressurized acetylene-rich solvent effluent 114 to a first heat exchanger 116. For example, pump 112 may be a rotary pump including an impeller, or alternatively may be any other suitable fluid pump such as a centrifugal pump, a positive displacement pump, etc.

System 100 further includes first heat exchanger 116 for receiving pressurized acetylene-rich solvent effluent 114 from pump 112 and a pressurized heated acetylene-lean solvent effluent 138 from a pump 136 as a heat transfer medium to generate a first heated pressurized acetylene-rich solvent stream 118. In other words, pressurized heated acetylene-lean solvent effluent 138 delivers the heat in first heat exchanger 116 to pressurized acetylene-rich solvent effluent 114 and generates first heated pressurized acetylene-rich solvent stream 118 having a temperature of from about 20° C. to about 200° C., and pressurized heated acetylene-lean solvent effluent 138 is likewise cooled against pressurized acetylene-rich solvent effluent 114 in first heat exchanger 116 to generate a cooled pressurized acetylene-lean solvent effluent 140 as discussed below. In some embodiments, first heat exchanger 116 may be a shell-and-tube, plate-fin, microchannel, spiral wound, or any other suitable heat exchanger.

System 100 further includes a second heat exchanger 120 for receiving first heated pressurized acetylene-rich solvent stream 118 from first heat exchanger 116 to generate a second heated pressurized acetylene-rich solvent stream 122. Although two heat exchangers are shown in heating pressurized acetylene-rich solvent effluent 114 and first heated pressurized acetylene-rich solvent stream 118 to a temperature sufficient to enter an acetylene hydrogenation reactor 126 (also referred to as “hydrogenation reactor”) as discussed below, this is merely illustrative and any number of heat exchangers are contemplated herein.

Second heat exchanger 120 can have a heat source that is provided by, for example, higher temperature fluid lines or a resistive or inductive heating element. In some embodiments, the fluid lines can include, for example, a heat transfer fluid including one or more of water (such as water provided to or from a boiler), steam, ethane, ethylene, propane, propylene, such as propylene from a unit downstream of acetylene hydrogenation reactor 126, or any other suitable fluid, and/or a stream that is associated with another portion within system 100. The heat transfer fluid can be provided at a temperature above the temperature of first heated pressurized acetylene-rich solvent stream 118 in order to transfer heat to first heated pressurized acetylene-rich solvent stream 118 and generates second heated pressurized acetylene-rich solvent stream 122 having a temperature of from about 20° C. to about 200° C. In some embodiments, second heat exchanger 120 can be an electric heater. In some embodiments, second heat exchanger 120 may be a shell-and-tube, plate-fin, microchannel, spiral wound, or any other suitable heat exchanger.

System 100 further includes acetylene hydrogenation reactor 126 for receiving second heated pressurized acetylene-rich solvent stream 122 and a hydrogen stream 124 in acetylene hydrogenation reactor 126. In some embodiments, second heated pressurized acetylene-rich solvent stream 122 is received in a bottom injection point of acetylene hydrogenation reactor 126. As one skilled in the art will appreciate, hydrogen stream 124 can be received in acetylene hydrogenation reactor 126 in one or more injection points. As shown in FIG. 1A, acetylene hydrogenation reactor 126 can be configured to have one or more injection points for receiving hydrogen streams 124-1, 124-2, 124-3 and 124-4. Thus, for example, acetylene hydrogenation reactor 126 can be configured to receive only hydrogen stream 124-1 or hydrogen stream 124-2 or hydrogen stream 124-3 or hydrogen stream 124-4 or any combination thereof.

Acetylene hydrogenation reactor 126 includes catalyst beds 128a, 128b, 128c and 128d for receiving second heated pressurized acetylene-rich solvent stream 122 and one or more of hydrogen streams 124-1, 124-2, 124-3 and 124-4. Second heated pressurized acetylene-rich solvent stream 122 may be contacted with one or more of hydrogen streams 124-1, 124-2, 124-3 and 124-4 in the presence of a hydrogenation catalyst in each of catalyst beds 128a-128d in acetylene hydrogenation reactor 126 to produce a hydrogenation effluent 132, which may include the constituents of second heated pressurized acetylene-rich solvent stream 122 and reaction products from the hydrogenation reaction and a heated acetylene-lean solvent effluent 134. The contacting of second heated pressurized acetylene-rich solvent stream 122 with one or more of hydrogen streams 124-1, 124-2, 124-3 and 124-4 in the presence of the hydrogenation catalyst may cause hydrogenation of at least a portion of acetylene in second heated pressurized acetylene-rich solvent stream 122 to produce hydrogenation effluent 132, which may have a reduced concentration of acetylene compared to second heated pressurized acetylene-rich solvent stream 122. For example, in acetylene hydrogenation reactor 126, in the presence of the hydrogenation catalyst, under hydrogenation reaction conditions, the hydrogen in one or more of hydrogen streams 124-1, 124-2, 124-3 and 124-4 reacts with the acetylene in second heated pressurized acetylene-rich solvent stream 122 to selectively produce ethylene.

Although four catalyst beds are shown in acetylene hydrogenation reactor 126, this is merely illustrative and any number of catalyst beds may be included in acetylene hydrogenation reactor 126. In some embodiments, acetylene hydrogenation reactor 126 may be a fixed bed reactor including catalyst beds 128a-128d as fixed catalyst beds of the hydrogenation catalyst. Heat exchangers 130a, 130b and 130c may be selectively positioned between each of catalyst beds 128a-128d of acetylene hydrogenation reactor 126 to assist in regulating the desired temperature of the hydrogenation reaction in each of catalyst beds 128b, 128c and 128d. Heat exchangers 130a, 130b and 130c can be any type of heat exchanger such as those described above for second heat exchanger 120.

Acetylene hydrogenation reactor 126 may include one or a plurality of temperature sensors, pressure sensors, flow meters, or combinations of these for measuring the temperature, pressure, or gas flow rates at one or a plurality of positions in acetylene hydrogenation reactor 126 (not shown). The temperature, pressure, and/or gas flow rate may be determined for one or more of catalyst beds 128a-128d of acetylene hydrogenation reactor 126 and/or for second heated pressurized acetylene-rich solvent stream 122 introduced to acetylene hydrogenation reactor 126. The method of operating acetylene hydrogenation reactor 126 may include determining the temperature of acetylene hydrogenation reactor 126, a temperature of second heated pressurized acetylene-rich solvent stream 122 passed to acetylene hydrogenation reactor 126, or both.

Hydrogen can be provided in one or more of hydrogen streams 124-1, 124-2, 124-3 and 124-4 at a variety of concentrations. The person of ordinary skill in the art will select an amount of hydrogen that provides the necessary reduction of acetylene, and, for example, to provide the desired amount of hydrogen for a subsequent process step. In some embodiments, the hydrogen is present in one or more of hydrogen streams 124-1, 124-2, 124-3 and 124-4 in an amount of at least about 5 mol. %, e.g., a range of from about 5 mol. % to about 100 mol. %. The hydrogen may come from a portion of a downstream reaction effluent, or hydrogen may be added to the process.

Hydrogen can also be provided in hydrogen stream and injected separately to each catalyst bed 128a-128d as hydrogen streams 124-1, 124-2, 124-3 and 124-4. In some embodiment, the total moles of hydrogen supplied to acetylene hydrogenation reactor 126 shall not exceed 10 times of the total moles of acetylene in the acetylene-rich solvent stream entered in acetylene hydrogenation reactor 126. In some embodiment, the total moles of hydrogen supplied to acetylene hydrogenation reactor 126 shall not exceed 5 times, or 2 times of the total moles of acetylene in the acetylene-rich solvent stream entered in acetylene hydrogenation reactor 126.

Suitable hydrogenation catalysts may be an acetylene hydrogenation catalyst that is a catalyst selective for hydrogenating acetylene. The hydrogenation catalyst may be any known catalyst for selectively hydrogenating acetylene. Commercial catalysts for acetylene hydrogenation are widely available, and the present disclosure is not limited to any specific composition recited herein. In some embodiments, a hydrogenation catalyst can include a hydrogenation metal in an amount between about 0.01 wt. % to about 5.0 wt. % on a support, wherein the hydrogenation metal is selected from a transition metal. In some embodiments, the metal can be platinum (Pt), palladium (Pd), rhodium (Rh), iridium (Ir), ruthenium (Ru), nickel (Ni), or a mixture thereof. In some embodiments, a transition metal is modified by one or more metals, selected from Group IB through IVA, such as zinc (Zn), indium (In), tin (Sn), lead (Pb), copper (Cu), silver (Ag), gold (Au), molybdenum (Mo), tungsten (W), titanium (Ti), niobium (Nb), ytterbium (Y), cobalt (Co) in an amount between about 0.01 and about 5 wt. %. Suitable supports include, for example, carbon materials such as activated carbon, carbon nanotubes, carbon fibers, etc.; aluminum oxides (aluminas); SiO₂, pure or doped with other metal oxides, synthetic or natural such as quartz; titanium silicate; CaCO₃; BaSO₄; and MgO. In some embodiments, supports include alpha-aluminas of various shapes and sizes (i.e., spheres, extrudates), with high degree of conversion to the alpha phase.

Suitable hydrogenation reaction conditions in acetylene hydrogenation reactor 126 include, for example, a temperature that may range between about 20° C. and about 250° C., or between about 40° C. and about 200° C., or between about 40° C. and about 120° C. In addition, acetylene hydrogenation reactor 126 can be operated at a high pressure which may range between approximately about 0.14 MPa (20 psig) and about 3.4 MPa (500 psig), or between about 1.0 MPa (150 psig) and about 2.8 MPa (400 psig). The liquid hour space velocity (LHSV) at the reactor inlet of acetylene hydrogenation reactor 126 can range between about 1 and about 100 h⁻¹, or between about 5 and about 50 h⁻¹, or about 5 and about 25 h⁻¹.

Hydrogenation effluent 132 exits acetylene hydrogenation reactor 126 as an overhead stream and is rich in ethylene and may contain other gases such as hydrogen and constituents of second heated pressurized acetylene-rich solvent stream 122. Hydrogenation effluent 132 may be passed to other processing and separation zones, the particulars of which are not necessary for an understanding and practicing of the present invention. For example, hydrogenation effluent 132 may be sent to a separation unit for extracting ethylene to send for further processing. Additionally, since hydrogenation effluent 132 may include hydrogen, a portion of hydrogenation effluent 132 may be recycled back to acetylene hydrogenation reactor 126 to provide hydrogen for the hydrogenation reactions. In some embodiments, hydrogenation effluent 132 may be passed to one or more other reactors for further processing.

As discussed above, contacting second heated pressurized acetylene-rich solvent stream 122 with one or more of hydrogen streams 124-1, 124-2, 124-3 and 124-4 in the presence of a hydrogenation catalyst in each of catalyst beds 128a-128d in acetylene hydrogenation reactor 126 likewise produces heated acetylene-lean solvent effluent 134 containing a reduced amount of acetylene relative to second heated pressurized acetylene-rich solvent stream 122. Heated acetylene-lean solvent effluent 134 exits acetylene hydrogenation reactor 126 at a top injection point of acetylene hydrogenation reactor 126 and at an elevated temperature, e.g., a temperature ranging from about 100° C. to about 200° C., and is sent to a pump 136. Pump 136 can be any suitable pump as discussed above for pump 112 for increasing the pressure of heated acetylene-lean solvent effluent 134 for sending pressurized heated acetylene-lean solvent effluent 138 to first heat exchanger 116.

As discussed above, first heat exchanger 116 receives pressurized acetylene-rich solvent effluent 114 from pump 112 and pressurized heated acetylene-lean solvent effluent 138 from pump 136 as a heat transfer medium. In other words, pressurized heated acetylene-lean solvent effluent 138 delivers the heat in first heat exchanger 116 to pressurized acetylene-rich solvent effluent 114 and generates first heated pressurized acetylene-rich solvent stream 118 and pressurized heated acetylene-lean solvent effluent 138 is likewise cooled against pressurized acetylene-rich solvent effluent 114 in first heat exchanger 116 to generate cooled pressurized acetylene-lean solvent effluent 140 having a temperature less than the temperature of pressurized heated acetylene-lean solvent effluent 138.

System 100 further includes a third heat exchanger 142 for receiving cooled pressurized acetylene-lean solvent effluent 140 from first heat exchanger 116 to generate cooled pressurized acetylene-lean solvent effluent 106. Although two heat exchangers are shown in cooling pressurized acetylene-rich solvent effluent 114 and cooled pressurized acetylene-lean solvent effluent 140 to a temperature sufficient to enter acetylene absorption unit 102, this is merely illustrative and any number of heat exchangers are contemplated herein. Third heat exchanger 142 can have a cooling source that is provided by, for example, lower temperature fluid lines. In some embodiments, the fluid lines can include, for example, a heat transfer fluid including one or more of water (such as water provided to or from a refrigerant), ethane, ethylene, propane, propylene, such as propylene from a unit downstream of acetylene hydrogenation reactor 126, or any other suitable fluid, and/or a stream that is associated with another portion within system 100. The heat transfer can be provided at a temperature below the temperature of cooled pressurized acetylene-lean solvent effluent 140 in order to remove heat from cooled pressurized acetylene-lean solvent effluent 140 and generate cooled pressurized acetylene-lean solvent effluent 106 having a temperature of from about 0° C. to about 50° C. In some embodiments, third heat exchanger 142 may be a shell-and-tube, plate-fin, microchannel, spiral wound, or any other suitable heat exchanger.

FIG. 1B illustrates an alternative non-limiting embodiment of second heated pressurized acetylene-rich solvent stream 122 and hydrogen stream 124 in a countercurrent flow as compared to second heated pressurized acetylene-rich solvent stream 122 and hydrogen stream 124 in a co-current flow illustrated in FIG. 1A. As shown in FIG. 1B, system 100 includes acetylene hydrogenation reactor 126 for receiving second heated pressurized acetylene-rich solvent stream 122 in a top injection point of acetylene hydrogenation reactor 126 and hydrogen stream 124 in acetylene hydrogenation reactor 126. As discussed above, hydrogen stream 124 can be received in acetylene hydrogenation reactor 126 in one or more injection points. As shown in FIG. 1B, acetylene hydrogenation reactor 126 can be configured to have one or more injection points for receiving hydrogen streams 124-1, 124-2, 124-3 and 124-4. Thus, for example, acetylene hydrogenation reactor 126 can be configured to receive only hydrogen stream 124-1 or hydrogen stream 124-2 or hydrogen stream 124-3 or hydrogen stream 124-4 or any combination thereof.

Hydrogen can be provided in one or more of hydrogen streams 124-1, 124-2, 124-3 and 124-4 at a variety of concentrations. The person of ordinary skill in the art will select an amount of hydrogen that provides the necessary reduction of acetylene, and, for example, to provide the desired amount of hydrogen for a subsequent process step. In some embodiments, the hydrogen is present in one or more of hydrogen streams 124-1, 124-2, 124-3 and 124-4 in an amount of at least about 5 mol. %, e.g., a range of from about 5 mol. % to about 100 mol. %. The hydrogen may come from a portion of a downstream reaction effluent, or hydrogen may be added to the process.

Hydrogen can also be provided in hydrogen stream and injected separately to each catalyst bed 128a-128d as hydrogen streams 124-1, 124-2, 124-3 and 124-4. In some embodiment, the total moles of hydrogen supplied to acetylene hydrogenation reactor 126 shall not exceed 10 times of the total moles of acetylene in the acetylene-rich solvent stream entered in acetylene hydrogenation reactor 126. In some embodiment, the total moles of hydrogen supplied to acetylene hydrogenation reactor 126 shall not exceed 5 times, or 2 times of the total moles of acetylene in the acetylene-rich solvent stream entered in acetylene hydrogenation reactor 126.

Second heated pressurized acetylene-rich solvent stream 122 flows downwards through catalyst beds 128a, 128b, 128c and 128d and heat exchangers 130a-130c in acetylene hydrogenation reactor 126 and hydrogen stream 124 flows upwards through catalyst beds 128a, 128b, 128c and 128d and heat exchangers 130a-130c in acetylene hydrogenation reactor 126 to produce hydrogenation effluent 132. In some embodiments, in operation, second heated pressurized acetylene-rich solvent stream 122 is a liquid stream while hydrogen stream 124 is a gas stream. When in a concurrent flow configuration as depicted in FIG. 1A, relatively most if not all of the entire acetylene hydrogenation reactor 126 is filled with liquid and one or more of hydrogen streams 124-1, 124-2, 124-3 and 124-4 moves up in acetylene hydrogenation reactor 126 as gas bubbles. When in a count-current flow as depicted in FIG. 1B, acetylene hydrogenation reactor 126 will be filled with gas from one or more of hydrogen streams 124-1, 124-2, 124-3 and 124-4 and the liquid is trickling down through catalyst beds 128a-128d.

Second heated pressurized acetylene-rich solvent stream 122 may be contacted with hydrogen stream 124 in the presence of a hydrogenation catalyst in each of catalyst beds 128a-128d in acetylene hydrogenation reactor 126 to produce hydrogenation effluent 132 as discussed above, which may include the constituents of second heated pressurized acetylene-rich solvent stream 122 and reaction products from the hydrogenation reaction and heated acetylene-lean solvent effluent 134. The contacting of second heated pressurized acetylene-rich solvent stream 122 with one or more of hydrogen streams 124-1, 124-2, 124-3 and 124-4 in the presence of the hydrogenation catalyst may cause hydrogenation of at least a portion of acetylene in second heated pressurized acetylene-rich solvent stream 122 to produce hydrogenation effluent 132, which may have a reduced concentration of acetylene compared to second heated pressurized acetylene-rich solvent stream 122. For example, in acetylene hydrogenation reactor 126, in the presence of the hydrogenation catalyst, under hydrogenation reaction conditions, the hydrogen in one or more of hydrogen streams 124-1, 124-2, 124-3 and 124-4 reacts with the acetylene in second heated pressurized acetylene-rich solvent stream 122 to selectively produce ethylene.

Hydrogen stream 124 may also be supplied and injected separately to each of catalyst beds 128a-128d as hydrogen streams 124-1, 124-2, 124-3 and 124-4 to effectively reduce hydrogen concentration in acetylene hydrogenation reactor 126 for improved reaction selectivity. For example, one of the by-reactions is the reaction of ethylene with hydrogen producing ethane. Thus, reducing the hydrogen concentration will in turn reduce this by-reaction.

Heated acetylene-lean solvent effluent 134 exits acetylene hydrogenation reactor 126 through a bottom exit point and is sent to pump 136, first and third heat exchangers 116 and 142 as discussed above to generate cooled pressurized acetylene-lean solvent effluent 106 for sending to acetylene absorption unit 102.

FIG. 1C illustrates a non-limiting alternative illustrative embodiment of the selective hydrogenation of acetylene in an acetylene-rich solvent stream to ethylene in a plurality of acetylene selective hydrogenation reactors operated in parallel with a solvent recycle loop. Starting with second heated pressurized acetylene-rich solvent stream 122 as produced in second heat exchanger 120 as described above with reference to FIG. 1A, second heated pressurized acetylene-rich solvent stream 122 is split into a third heated pressurized acetylene-rich solvent stream 122a and a fourth heated pressurized acetylene-rich solvent stream 122b.

Third heated pressurized acetylene-rich solvent stream 122a enters acetylene hydrogenation reactor 126 in a bottom injection point and flows upwards through catalyst beds 128a, 128b, 128c and 128d and heat exchangers 130a-130c with hydrogen stream 124. Third heated pressurized acetylene-rich solvent stream 122a may be contacted with one or more of hydrogen streams 124-1, 124-2, 124-3 and 124-4 in the presence of the hydrogenation catalyst in each of catalyst beds 128a-128d in acetylene hydrogenation reactor 126 to produce hydrogenation effluent 132, which may include the constituents of third heated pressurized acetylene-rich solvent stream 122a and reaction products from the hydrogenation reaction and heated acetylene-lean solvent effluent 134. The contacting of third heated pressurized acetylene-rich solvent stream 122a with one or more of hydrogen streams 124-1, 124-2, 124-3 and 124-4 in the presence of the hydrogenation catalyst is described above with reference to FIG. 1A.

Hydrogenation effluent 132 exits acetylene hydrogenation reactor 126 as an overhead stream and is rich in ethylene and may contain other gases such as hydrogen and constituents of second heated pressurized acetylene-rich solvent stream 122. Hydrogenation effluent 132 may be passed to other processing and separation zones, the particulars of which are not necessary for an understanding and practicing of the present invention. For example, hydrogenation effluent 132 may be sent to a separation unit for extracting ethylene to send for further processing. Additionally, since hydrogenation effluent 132 may include hydrogen, a portion of hydrogenation effluent 132 may be recycled back to acetylene hydrogenation reactor 126 to provide hydrogen for the hydrogenation reactions.

As shown in FIG. 1C, system 100 further includes an acetylene hydrogenation reactor 146 operated in parallel with acetylene hydrogenation reactor 126. Although only two acetylene hydrogenation reactors are shown in this particular embodiment, it is contemplated that system 100 can include any number of acetylene hydrogenation reactors such from 2 to 10 or greater. Acetylene hydrogenation reactor 146 is configured to receive fourth heated pressurized acetylene-rich solvent stream 122b in a bottom injection point of acetylene hydrogenation reactor 146a and a hydrogen stream 148 in one or more injection points of acetylene hydrogenation reactor 146 as discussed above with regard to hydrogen stream 124. As shown in FIG. 1C, acetylene hydrogenation reactor 146 can be configured to have one or more injection points for receiving hydrogen streams 148-1, 148-2, 148-3 and 148-4. Thus, for example, acetylene hydrogenation reactor 146 can be configured to receive only hydrogen stream 148-1 or hydrogen stream 148-2 or hydrogen stream 148-3 or hydrogen stream 148-4 or any combination thereof.

Acetylene hydrogenation reactor 146 includes catalyst beds 150a, 150b, 150c and 150d for receiving fourth heated pressurized acetylene-rich solvent stream 122b and hydrogen stream 148. Fourth heated pressurized acetylene-rich solvent stream 122b may be contacted with one or more of hydrogen streams 148-1, 148-2, 148-3 and 148-4 in the presence of a hydrogenation catalyst in each of catalyst beds 150a-150d in acetylene hydrogenation reactor 146 to produce a hydrogenation effluent 154, which may include the constituents of fourth heated pressurized acetylene-rich solvent stream 122b and reaction products from the hydrogenation reaction and a heated acetylene-lean solvent effluent 158. The contacting of fourth heated pressurized acetylene-rich solvent stream 122b with hydrogen stream 148 in the presence of the hydrogenation catalyst may cause hydrogenation of at least a portion of acetylene in fourth heated pressurized acetylene-rich solvent stream 122b to produce hydrogenation effluent 154, which may have a reduced concentration of acetylene compared to fourth heated pressurized acetylene-rich solvent stream 122b. For example, in acetylene hydrogenation reactor 146, in the presence of the hydrogenation catalyst, under hydrogenation reaction conditions, the hydrogen in one or more of hydrogen streams 148-1, 148-2, 148-3 and 148-4 reacts with the acetylene in fourth heated pressurized acetylene-rich solvent stream 122b to selectively produce ethylene.

Although four catalyst beds are shown in acetylene hydrogenation reactor 146, this is merely illustrative and any number of catalyst beds may be included in acetylene hydrogenation reactor 146. In some embodiments, acetylene hydrogenation reactor 146 may be a fixed bed reactor including catalyst beds 150a-150d as fixed catalyst beds of the hydrogenation catalyst. Heat exchangers 152a, 152b and 152c may be selectively positioned between each of catalyst beds 150a-150d of the acetylene hydrogenation reactor 146 to assist in regulating the desired temperature of the hydrogenation reaction in each of catalyst beds 150b, 150c and 150d. Heat exchangers 152a, 152b and 152c can be any type of heat exchanger such as those described above for second heat exchanger 120.

Acetylene hydrogenation reactor 146 may include one or a plurality of temperature sensors, pressure sensors, flow meters, or combinations of these for measuring the temperature, pressure, or gas flow rates at one or a plurality of positions in acetylene hydrogenation reactor 146 (not shown) as described above for acetylene hydrogenation reactor 126.

Hydrogen can be provided in one or more of hydrogen streams 148-1, 148-2, 148-3 and 148-4 at a variety of similar concentrations as described above for hydrogen stream 124. Suitable hydrogenation catalysts may be an acetylene hydrogenation catalyst that is a catalyst selective for hydrogenating acetylene as discussed above.

Suitable hydrogenation reaction conditions in acetylene hydrogenation reactor 146 include, for example, a temperature that may range between about 20° C. and about 250° C., or between about 40° C. and about 200° C., or between about 40° C. and about 120° C. In addition, acetylene hydrogenation reactor 146 can be operated at a high pressure which may range between approximately about 0.14 MPa (20 psig) and about 3.4 MPa (500 psig), or between about 1.0 MPa (150 psig) and about 2.8 MPa (400 psig). The liquid hour space velocity (LHSV) at the reactor inlet of acetylene hydrogenation reactor 146 can range between about 1 and about 100 h⁻¹, or between about 5 and about 50 h⁻¹, or about 5 and about 25 h⁻¹.

Hydrogenation effluent 154 exits acetylene hydrogenation reactor 146 as an overhead stream and is rich in ethylene and may contain other gases such as hydrogen and constituents of fourth heated pressurized acetylene-rich solvent stream 122b. Hydrogenation effluent 154 may be passed to other processing and separation zones, the particulars of which are not necessary for an understanding and practicing of the present invention. For example, hydrogenation effluent 154 may be sent to a separation unit for extracting ethylene to send for further processing. Additionally, since hydrogenation effluent 154 may include hydrogen, a portion of hydrogenation effluent 154 may be recycled back to acetylene hydrogenation reactor 146 to provide hydrogen for the hydrogenation reactions. In some embodiments, hydrogenation effluent 154 may be passed to one or more other reactors for further processing.

In non-limiting illustrative embodiments, acetylene hydrogenation reactor 126 and acetylene hydrogenation reactor 146 can be operated in a manner by cycling between a normal operation mode and a catalyst regeneration mode. In some embodiments, during regeneration, catalyst beds 128a-128d in acetylene hydrogenation reactor 126 and catalyst beds 150a-150d in acetylene hydrogenation reactor 146 are independently purged to remove substantially all liquid solvent present in the catalyst beds, followed by hot stripping with steam to remove, for example, any green oil or other materials responsible for the catalyst deactivation.

In some embodiments, an oxidizing stream 144 and an oxidizing stream 156 can be introduced into respective acetylene hydrogenation reactor 126 and acetylene hydrogenation reactor 146 to regenerate any spent hydrogenation catalyst present in respective acetylene hydrogenation reactor 126 and acetylene hydrogenation reactor 146. For example, during the hydrogenation process, impurities such as heavy hydrocarbons and coke are deposited on the hydrogenation catalyst causing the hydrogenation catalyst to become spent hydrogenation catalyst. These impurities can be burned from the spent hydrogenation catalyst by exposing the spent hydrogenation catalyst to respective oxidizing streams 144 and 156 at appropriate high temperature and time duration conditions to burn off and remove substantially all these impurities from the catalyst.

In some embodiments, oxidizing stream 144 and oxidizing stream 156 can independently include, for example, an inert gas/air such as air, oxygen, nitrogen, methane, or combinations thereof or a steam/air mixture. In an illustrative embodiment, a temperature can range from about 500° C. to about 800° C., and a time period can range from about 1 hour to about 24 hours. Accordingly, regenerating the spent hydrogenation catalyst generally comprises combustion of the spent hydrogenation catalyst in an oxidizing atmosphere to burn the impurities such as heavy hydrocarbons and coke deposits and redisperse active metal on the catalyst particles.

In some embodiments, the coke burn causes the spent hydrogenation catalyst to be heated to an elevated temperature, e.g., a temperature of from about 500° C. to about 800° C., to provide a heated regenerated catalyst relatively free or free of coke wherein the catalyst particles are heated, and a heated gas effluent (not shown). In some embodiments the heated gas effluent is regenerator flue gas composed of, for example, carbon dioxide and nitrogen.

Referring back to FIG. 1C, heated acetylene-lean solvent effluent 134 and heated acetylene-lean solvent effluent 158 exit respective acetylene hydrogenation reactor 126 and acetylene hydrogenation reactor 146 at a top injection point and at elevated temperatures, e.g., a temperature ranging from about 100° C. to about 250° C. Heated acetylene-lean solvent effluent 134 and heated acetylene-lean solvent effluent 158 are then combined into a combined heated acetylene-lean solvent effluent 160 and is sent to a pump 136 as described above with reference to FIG. 1A for increasing the pressure of combined heated acetylene-lean solvent effluent 160 for sending pressurized heated acetylene-lean solvent effluent 138 to first heat exchanger 116.

As discussed above, first heat exchanger 116 receives pressurized acetylene-rich solvent effluent 114 from pump 112 and pressurized heated acetylene-lean solvent effluent 138 from pump 136 as a heat transfer medium. In other words, pressurized heated acetylene-lean solvent effluent 138 delivers the heat in first heat exchanger 116 to pressurized acetylene-rich solvent effluent 114 and generates first heated pressurized acetylene-rich solvent stream 118 and pressurized heated acetylene-lean solvent effluent 138 is likewise cooled against pressurized acetylene-rich solvent effluent 114 in first heat exchanger 116 to generate cooled pressurized acetylene-lean solvent effluent 140. Cooled pressurized acetylene-lean solvent effluent 140 is then sent to third heat exchanger 142 to generate cooled pressurized acetylene-lean solvent effluent 106.

According to an aspect of the disclosure, a system, comprises:
an absorption column configured to receive an acetylene-rich gas stream flowing upwards and a cooled acetylene-lean solvent stream flowing downwards to generate an acetylene-lean gas effluent and an acetylene-rich solvent effluent,
one or more heat exchangers in fluid communication with the absorption column for receiving the acetylene-rich solvent effluent, the one or more heat exchangers being configured to transfer heat to the acetylene-rich solvent effluent to generate a heated acetylene-rich solvent stream, and
one or more hydrogenation reactors, in fluid communication with the one or more heat exchangers, each comprising one or more catalyst beds, wherein at least a first one of the one or more hydrogenation reactors is configured to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

In one or more additional non-limiting illustrative embodiments, as may be combined with one or more of the preceding paragraphs, the one or more heat exchangers comprise a first heat exchanger configured to receive the acetylene-rich solvent effluent and the first acetylene-lean solvent effluent to transfer heat from the first acetylene-lean solvent effluent to the acetylene-rich solvent effluent to generate the heated acetylene-rich solvent stream and the cooled acetylene-lean solvent stream.

In one or more additional non-limiting illustrative embodiments, as may be combined with one or more of the preceding paragraphs, the one or more heat exchangers further comprise a second heat exchanger configured to receive the heated acetylene-rich solvent stream to transfer additional heat to the heated acetylene-rich solvent stream for passing to the first one of the one or more hydrogenation reactors.

In one or more additional non-limiting illustrative embodiments, as may be combined with one or more of the preceding paragraphs, the one or more heat exchangers further comprise a second heat exchanger configured to receive the cooled acetylene-lean solvent stream to further cool the cooled acetylene-lean solvent stream for passing to the absorption column.

In one or more additional non-limiting illustrative embodiments, as may be combined with one or more of the preceding paragraphs, the system further comprises a first pump configured to increase the pressure of the acetylene-rich solvent effluent prior to sending to the first heat exchanger, and a second pump configured to increase the pressure of the first acetylene-lean solvent effluent prior to sending to the first heat exchanger.

In one or more additional non-limiting illustrative embodiments, as may be combined with one or more of the preceding paragraphs, the first one of the one or more hydrogenation reactors comprises one or more other heat exchangers each positioned between respective catalyst beds.

In one or more additional non-limiting illustrative embodiments, as may be combined with one or more of the preceding paragraphs, the first one of the one or more hydrogenation reactors is further configured to receive the heated acetylene-rich solvent stream in a bottom end of the first one of the one or more hydrogenation reactors flowing upwards to generate the first hydrogenation effluent and the first acetylene-lean solvent effluent, wherein the first hydrogenation effluent comprising ethylene and the first acetylene-lean solvent effluent exit from a first top position and a second top position, respectively, of the first one of the one or more hydrogenation reactors.

In one or more additional non-limiting illustrative embodiments, as may be combined with one or more of the preceding paragraphs, the first one of the one or more hydrogenation reactors is further configured to receive the heated acetylene-rich solvent stream in a first top position of the first one of the one or more hydrogenation reactors flowing downwards to generate the first hydrogenation effluent and the first acetylene-lean solvent effluent, wherein the first hydrogenation effluent comprising ethylene exits through a second top position of the first one of the one or more hydrogenation reactors and the first acetylene-lean solvent effluent exits from a bottom end of the first one of the one or more hydrogenation reactors.

In one or more additional non-limiting illustrative embodiments, as may be combined with one or more of the preceding paragraphs, the heated acetylene-rich solvent stream is split into a first heated acetylene-rich solvent stream and a second heated acetylene-rich solvent stream and the first one of the one or more hydrogenation reactors is configured to convert at least a portion of acetylene in the first heated acetylene-rich solvent stream to ethylene and a second one of the one or more hydrogenation reactors, parallel to the first one of the one or more hydrogenation reactors, is configured to convert at least a portion of acetylene in the second heated acetylene-rich solvent stream to ethylene in the presence of a second hydrogenation catalyst and hydrogen under second hydrogenation reaction conditions to generate a second hydrogenation effluent comprising ethylene and a second acetylene-lean solvent effluent.

In one or more additional non-limiting illustrative embodiments, as may be combined with one or more of the preceding paragraphs, the first one of the one or more hydrogenation reactors and the second one of the one or more hydrogenation reactors are each further configured to receive an oxidizing stream to respectively regenerate the first hydrogenation catalyst and the second hydrogenation catalyst.

According to another aspect of the disclosure, a continuous process comprises:
passing an acetylene-rich gas stream to an absorption column flowing upwards and a cooled acetylene-lean solvent stream flowing downwards to generate an acetylene-lean gas effluent and an acetylene-rich solvent effluent,
passing the acetylene-rich solvent effluent through one or more heat exchangers to transfer heat to the acetylene-rich solvent effluent and generate a heated acetylene-rich solvent stream, and
passing the heated acetylene-rich solvent stream to at least a first one of one or more hydrogenation reactors each comprising one or more catalyst beds to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

In one or more additional non-limiting illustrative embodiments, as may be combined with one or more of the preceding paragraphs, passing the acetylene-rich solvent effluent through the one or more heat exchangers comprises passing the acetylene-rich solvent effluent and the first acetylene-lean solvent effluent to a first heat exchanger to transfer heat from the first acetylene-lean solvent effluent to the acetylene-rich solvent effluent to generate the heated acetylene-rich solvent stream and the cooled acetylene-lean solvent stream.

In one or more additional non-limiting illustrative embodiments, as may be combined with one or more of the preceding paragraphs, the continuous process further comprises passing the heated acetylene-rich solvent stream to a second heat exchanger to transfer additional heat to the heated acetylene-rich solvent stream for passing to the first one of the one or more hydrogenation reactors.

In one or more additional non-limiting illustrative embodiments, as may be combined with one or more of the preceding paragraphs, the continuous process further comprises passing the cooled acetylene-lean solvent stream to a second heat exchanger to further cool the cooled acetylene-lean solvent stream for passing to the absorption column.

In one or more additional non-limiting illustrative embodiments, as may be combined with one or more of the preceding paragraphs, the continuous process further comprises passing the acetylene-rich solvent effluent to a first pump to increase the pressure of the acetylene-rich solvent effluent prior to sending to the first heat exchanger, and passing the first acetylene-lean solvent effluent to a second pump to increase the pressure of the first acetylene-lean solvent effluent prior to sending to the first heat exchanger.

In one or more additional non-limiting illustrative embodiments, as may be combined with one or more of the preceding paragraphs, the continuous process further comprises passing the heated acetylene-rich solvent stream to a bottom end of the first one of the one or more hydrogenation reactors flowing upwards to generate the first hydrogenation effluent and the first acetylene-lean solvent effluent, wherein the first hydrogenation effluent comprising ethylene and the first acetylene-lean solvent effluent exit from a top position of the first one of the one or more hydrogenation reactors.

In one or more additional non-limiting illustrative embodiments, as may be combined with one or more of the preceding paragraphs, the continuous process further comprises passing the heated acetylene-rich solvent stream to a first top position of the first one of the one or more hydrogenation reactors flowing downwards to generate the first hydrogenation effluent and the first acetylene-lean solvent effluent, wherein the first hydrogenation effluent comprising ethylene exits through a second top position of the first one of the one or more hydrogenation reactors and the first acetylene-lean solvent effluent exits from a bottom end of the first one of the one or more hydrogenation reactors.

In one or more additional non-limiting illustrative embodiments, as may be combined with one or more of the preceding paragraphs, the continuous process further comprising:
splitting the heated acetylene-rich solvent stream into a first heated acetylene-rich solvent stream and a second heated acetylene-rich solvent stream,
passing the first heated acetylene-rich solvent stream to the first one of the one or more hydrogenation reactors to convert at least a portion of acetylene in the first heated acetylene-rich solvent stream to ethylene, and
passing the second heated acetylene-rich solvent stream to a second one of the one or more hydrogenation reactors, parallel to the first one of the one or more hydrogenation reactors, to convert at least a portion of acetylene in the second heated acetylene-rich solvent stream to ethylene in the presence of a second hydrogenation catalyst and hydrogen under second hydrogenation reaction conditions to generate a second hydrogenation effluent comprising ethylene and a second acetylene-lean solvent effluent.

In one or more additional non-limiting illustrative embodiments, as may be combined with one or more of the preceding paragraphs, the continuous process further comprises introducing a first oxidizing stream to the first one of the one or more hydrogenation reactors to regenerate the first hydrogenation catalyst and introducing a second oxidizing stream to the second one of the one or more hydrogenation reactors to regenerate the second hydrogenation catalyst.

According to yet another aspect of the disclosure, a continuous process comprises:
receiving, in a heat exchanger, an acetylene-rich solvent effluent from an absorption column, and a first acetylene-lean solvent effluent from a hydrogenation reactor to transfer heat from the first acetylene-lean solvent effluent to the acetylene-rich solvent effluent and generate a heated acetylene-rich solvent stream and a cooled acetylene-lean solvent stream, and
passing the heated acetylene-rich solvent stream to the hydrogenation reactor comprising one or more catalyst beds to convert at least a portion of acetylene in the heated acetylene-rich solvent stream to ethylene in the presence of a hydrogenation catalyst and hydrogen under hydrogenation reaction conditions to generate a hydrogenation effluent comprising ethylene and a second acetylene-lean solvent effluent,
wherein the first acetylene-lean solvent effluent and the second acetylene-lean solvent effluent from the hydrogenation reactor and the acetylene-rich solvent effluent from the absorption column operate in a continuous solvent recycle loop.

In one or more additional non-limiting illustrative embodiments, as may be combined with one or more of the preceding paragraphs, the continuous process further comprises passing the heated acetylene-rich solvent stream to another heat exchanger to transfer additional heat to the heated acetylene-rich solvent stream for passing to the hydrogenation reactor.

In one or more additional non-limiting illustrative embodiments, as may be combined with one or more of the preceding paragraphs, the continuous process further comprises passing the cooled acetylene-lean solvent stream to another heat exchanger to further cool the cooled acetylene-lean solvent stream for passing to the absorption column.

In one or more additional non-limiting illustrative embodiments, as may be combined with one or more of the preceding paragraphs, the continuous process further comprises passing the acetylene-rich solvent effluent to a first pump to increase the pressure of the acetylene-rich solvent effluent prior to sending to the heat exchanger, and passing the first acetylene-lean solvent effluent to a second pump to increase the pressure of the first acetylene-lean solvent effluent prior to sending to the heat exchanger.

In one or more additional non-limiting illustrative embodiments, as may be combined with one or more of the preceding paragraphs, the continuous process further comprises passing the heated acetylene-rich solvent stream to a bottom end of the hydrogenation reactor flowing upwards to generate the hydrogenation effluent and the first acetylene-lean solvent effluent, wherein the hydrogenation effluent comprising ethylene and the first acetylene-lean solvent effluent exit from a top position of the hydrogenation reactor.

In one or more additional non-limiting illustrative embodiments, as may be combined with one or more of the preceding paragraphs, the continuous process further comprises passing the heated acetylene-rich solvent stream to a first top position of the hydrogenation reactor flowing downwards to generate the hydrogenation effluent and the first acetylene-lean solvent effluent, wherein the hydrogenation effluent comprising ethylene exits through a second top position of the hydrogenation reactor and the first acetylene-lean solvent effluent exits from a bottom end of the hydrogenation reactor.

In one or more additional non-limiting illustrative embodiments, as may be combined with one or more of the preceding paragraphs, the continuous process further comprises:
splitting the heated acetylene-rich solvent stream into a first heated acetylene-rich solvent stream and a second heated acetylene-rich solvent stream,
passing the first heated acetylene-rich solvent stream to the hydrogenation reactor to convert at least a portion of acetylene in the first heated acetylene-rich solvent stream to ethylene, and
passing the second heated acetylene-rich solvent stream to another hydrogenation reactor, parallel to the hydrogenation reactor, to convert at least a portion of acetylene in the second heated acetylene-rich solvent stream to ethylene in the presence of a second hydrogenation catalyst and hydrogen under second hydrogenation reaction conditions to generate a second hydrogenation effluent comprising ethylene and a second acetylene-lean solvent effluent.

In one or more additional non-limiting illustrative embodiments, as may be combined with one or more of the preceding paragraphs, the continuous process further comprises introducing a first oxidizing stream to the hydrogenation reactor to regenerate the first hydrogenation catalyst and introducing a second oxidizing stream to the other hydrogenation reactor to regenerate the second hydrogenation catalyst.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present disclosure and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A system, comprising:
an absorption column configured to receive an acetylene-rich gas stream flowing upwards and a cooled acetylene-lean solvent stream flowing downwards to generate an acetylene-lean gas effluent and an acetylene-rich solvent effluent;
one or more heat exchangers in fluid communication with the absorption column for receiving the acetylene-rich solvent effluent, the one or more heat exchangers being configured to transfer heat to the acetylene-rich solvent effluent to generate a heated acetylene-rich solvent stream; and
one or more hydrogenation reactors, in fluid communication with the one or more heat exchangers, each comprising one or more catalyst beds, wherein at least a first one of the one or more hydrogenation reactors is configured to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

2. The system according to claim 1, wherein the one or more heat exchangers comprise a first heat exchanger configured to receive the acetylene-rich solvent effluent and the first acetylene-lean solvent effluent to transfer heat from the first acetylene-lean solvent effluent to the acetylene-rich solvent effluent to generate the heated acetylene-rich solvent stream and the cooled acetylene-lean solvent stream.

3. The system according to claim 2, wherein the one or more heat exchangers further comprise a second heat exchanger configured to receive the heated acetylene-rich solvent stream to transfer additional heat to the heated acetylene-rich solvent stream for passing to the first one of the one or more hydrogenation reactors.

4. The system according to claim 2, wherein the one or more heat exchangers further comprise a second heat exchanger configured to receive the cooled acetylene-lean solvent stream to further cool the cooled acetylene-lean solvent stream for passing to the absorption column.

5. The system according to claim 2, further comprising a first pump configured to increase the pressure of the acetylene-rich solvent effluent prior to sending to the first heat exchanger, and a second pump configured to increase the pressure of the first acetylene-lean solvent effluent prior to sending to the first heat exchanger.

6. The system according to claim 1, wherein the first one of the one or more hydrogenation reactors comprises one or more other heat exchangers each positioned between respective catalyst beds.

7. The system according to claim 1, wherein the first one of the one or more hydrogenation reactors is further configured to receive the heated acetylene-rich solvent stream in a bottom end of the first one of the one or more hydrogenation reactors flowing upwards to generate the first hydrogenation effluent and the first acetylene-lean solvent effluent, wherein the first hydrogenation effluent comprising ethylene and the first acetylene-lean solvent effluent exit from a first top position and a second top position, respectively, of the first one of the one or more hydrogenation reactors.

8. The system according to claim 1, wherein the first one of the one or more hydrogenation reactors is further configured to receive the heated acetylene-rich solvent stream in a first top position of the first one of the one or more hydrogenation reactors flowing downwards to generate the first hydrogenation effluent and the first acetylene-lean solvent effluent, wherein the first hydrogenation effluent comprising ethylene exits through a second top position of the first one of the one or more hydrogenation reactors and the first acetylene-lean solvent effluent exits from a bottom end of the first one of the one or more hydrogenation reactors.

9. The system according to claim 1, wherein the heated acetylene-rich solvent stream is split into a first heated acetylene-rich solvent stream and a second heated acetylene-rich solvent stream and the first one of the one or more hydrogenation reactors is configured to convert at least a portion of acetylene in the first heated acetylene-rich solvent stream to ethylene and a second one of the one or more hydrogenation reactors, parallel to the first one of the one or more hydrogenation reactors, is configured to convert at least a portion of acetylene in the second heated acetylene-rich solvent stream to ethylene in the presence of a second hydrogenation catalyst and hydrogen under second hydrogenation reaction conditions to generate a second hydrogenation effluent comprising ethylene and a second acetylene-lean solvent effluent.

10. The system according to claim 9, wherein the first one of the one or more hydrogenation reactors and the second one of the one or more hydrogenation reactors are each further configured to receive an oxidizing stream to respectively regenerate the first hydrogenation catalyst and the second hydrogenation catalyst.

11. A continuous process, comprising:
passing an acetylene-rich gas stream to an absorption column flowing upwards and a cooled acetylene-lean solvent stream flowing downwards to generate an acetylene-lean gas effluent and an acetylene-rich solvent effluent;
passing the acetylene-rich solvent effluent through one or more heat exchangers to transfer heat to the acetylene-rich solvent effluent and generate a heated acetylene-rich solvent stream; and
passing the heated acetylene-rich solvent stream to at least a first one of one or more hydrogenation reactors each comprising one or more catalyst beds to convert at least a portion of acetylene in the heated acetylene-rich solvent stream to ethylene in the presence of a first hydrogenation catalyst and hydrogen under first hydrogenation reaction conditions to generate a first hydrogenation effluent comprising ethylene and a first acetylene-lean solvent effluent.

12. The continuous process according to claim 11, wherein passing the acetylene-rich solvent effluent through the one or more heat exchangers comprises passing the acetylene-rich solvent effluent and the first acetylene-lean solvent effluent to a first heat exchanger to transfer heat from the first acetylene-lean solvent effluent to the acetylene-rich solvent effluent to generate the heated acetylene-rich solvent stream and the cooled acetylene-lean solvent stream.

13. The continuous process according to claim 12, further comprising passing the heated acetylene-rich solvent stream to a second heat exchanger to transfer additional heat to the heated acetylene-rich solvent stream for passing to the first one of the one or more hydrogenation reactors.

14. The continuous process according to claim 12, further comprising passing the cooled acetylene-lean solvent stream to a second heat exchanger to further cool the cooled acetylene-lean solvent stream for passing to the absorption column.

15. The continuous process according to claim 12, further comprising passing the acetylene-rich solvent effluent to a first pump to increase the pressure of the acetylene-rich solvent effluent prior to sending to the first heat exchanger, and passing the first acetylene-lean solvent effluent to a second pump to increase the pressure of the first acetylene-lean solvent effluent prior to sending to the first heat exchanger.

16. The continuous process according to claim 11, further comprising passing the heated acetylene-rich solvent stream to a bottom end of the first one of the one or more hydrogenation reactors flowing upwards to generate the first hydrogenation effluent and the first acetylene-lean solvent effluent, wherein the first hydrogenation effluent comprising ethylene and the first acetylene-lean solvent effluent exit from a top position of the first one of the one or more hydrogenation reactors.

17. The continuous process according to claim 11, further comprising passing the heated acetylene-rich solvent stream to a first top position of the first one of the one or more hydrogenation reactors flowing downwards to generate the first hydrogenation effluent and the first acetylene-lean solvent effluent, wherein the first hydrogenation effluent comprising ethylene exits through a second top position of the first one of the one or more hydrogenation reactors and the first acetylene-lean solvent effluent exits from a bottom end of the first one of the one or more hydrogenation reactors.

18. The continuous process according to claim 11, further comprising:
splitting the heated acetylene-rich solvent stream into a first heated acetylene-rich solvent stream and a second heated acetylene-rich solvent stream;
passing the first heated acetylene-rich solvent stream to the first one of the one or more hydrogenation reactors to convert at least a portion of acetylene in the first heated acetylene-rich solvent stream to ethylene; and
passing the second heated acetylene-rich solvent stream to a second one of the one or more hydrogenation reactors, parallel to the first one of the one or more hydrogenation reactors, to convert at least a portion of acetylene in the second heated acetylene-rich solvent stream to ethylene in the presence of a second hydrogenation catalyst and hydrogen under second hydrogenation reaction conditions to generate a second hydrogenation effluent comprising ethylene and a second acetylene-lean solvent effluent.

19. The continuous process according to claim 18, further comprising introducing a first oxidizing stream to the first one of the one or more hydrogenation reactors to regenerate the first hydrogenation catalyst and introducing a second oxidizing stream to the second one of the one or more hydrogenation reactors to regenerate the second hydrogenation catalyst.

20. A continuous process, comprising:
receiving, in a heat exchanger, an acetylene-rich solvent effluent from an absorption column, and a first acetylene-lean solvent effluent from a hydrogenation reactor to transfer heat from the first acetylene-lean solvent effluent to the acetylene-rich solvent effluent and generate a heated acetylene-rich solvent stream and a cooled acetylene-lean solvent stream; and
passing the heated acetylene-rich solvent stream to the hydrogenation reactor comprising one or more catalyst beds to convert at least a portion of acetylene in the heated acetylene-rich solvent stream to ethylene in the presence of a hydrogenation catalyst and hydrogen under hydrogenation reaction conditions to generate a hydrogenation effluent comprising ethylene and a second acetylene-lean solvent effluent;
wherein the first acetylene-lean solvent effluent and the second acetylene-lean solvent effluent from the hydrogenation reactor and the acetylene-rich solvent effluent from the absorption column operate in a continuous solvent recycle loop.
